(19) United States
(12) Patent Application Publication
(10) Pub. No.: US 2012/0277603 A1
(43) Pub. Date: Nov. 1, 2012

(54) Device and Method for Detecting and reporting of a stress condition of a person

(76) Inventors: Martin Camenzind, Wil (CH); Markus Weder, Flawil (CH); Philipp Bachmann, Kusnacht (CH)

(21) Appl. No.: 13/094,046

(22) Filed: Apr. 26, 2011

(51) Int. Cl. A61B 5/02

(52) U.S. Cl. 600/500

(57) ABSTRACT

For easy determination of the current stress condition of a person, a device is proposed that detects the pulse rate and therefrom additionally determines the heart rate variability—preferably by means of a fast Fourier transform. In addition, at least one parameter related to the history of one of the two values mentioned above shall be used. Preferably, the deviations of the cardiac frequency and of the heart rate variability from corresponding norm parameters are integrated and thus used as an additional stress indicator.

### TECHNICAL FIELD

The invention relates to a device and to a method for detecting and reporting of a stress condition of a person.

### PRIOR ART

Using the pulse rate or the heart rate variability for the detection of stress conditions has been known for a long time. Thereby the interval between two heart beats is defined—in the sense of the present invention—as the time between the onsets of two contractions of the cardiac chambers. This onset of the chamber contraction shows up in the electrocardiogram (ECG) as the R wave. The distance between two R waves is usually denoted RR interval. After having averaged over a defined number of RR intervals, the heart rate can be determined by calculation. The individual values of the RR intervals vary around the mean value thus obtained. Thereby the variations can change from beat to beat. The variation is usually termed as heart rate variability (HRV). In principle, the heart rate can also be determined by a pressure measurement carried out on an artery.

Physiologically, the heart rate variability (HRV) depends on the ability of the human organism to adapt the rate of the cardiac rhythm. Variations of the heart rate, i.e. variations of the temporal interval between two heart beats, can occur in a resting state, in which case they are mostly spontaneous, but also in the course of specific variations of the surrounding conditions, e.g. under stress. A healthy organism continuously adapts the heart beat rate to the current conditions via physiological regulation pathways of the vegetative nervous system. Therefore, physical or psychological stress usually results in an increase of the heart frequency which ordinarily decreases again upon relief and relaxation. Thereby, a good adaptability to stress results in a higher variability of the heart rate. Under chronic stress burden, the adaptability is reduced. In this respect, it is known that the heart rate variability taken by itself already provides a certain—albeit still very unreliable—indicator for the current stress burden and the ability of a person to cope with stress.

Several methods for determining the stress condition of a person have been proposed in the prior art, including the proposal to use further measurement parameters in addition to the pulse rate. Thus, DE 103 19 361 A1 proposes to use the pulse wave latency in addition to the heart rate variability.

Regarding the analysis of the heart rate variability, reference is made to DE 100 06 154 A1, DE 10 2006 039 957 A1, and also to DE 10 2008 030 956 A1 and EP 1 156 851 B1, wherein the person skilled in the art can find various determination methods.

### DESCRIPTION OF THE INVENTION

It is an object of the invention to provide a device and a corresponding method for detecting and reporting of a stress condition of a person having a higher reliability as compared to the prior art.

The object of the invention is achieved according to a first aspect by a method for detecting and reporting of a stress condition of a person, the method comprising the following steps:
continuously acquiring the data of at least one body-physiological reference variable, wherein the at least one body-physiological reference variable comprises at least the pulse function of the person,
continuously processing the data of the at least one body-physiological reference variable to obtain a current status function of the person,
comparing the current status function of the person thus obtained with an alert criterion, whereby
at least one current pulse frequency P and a current heart rate variability HRV are continuously formed from the body-physiological reference variable, whereby
the status function is a function of the current pulse frequency P and of the current heart rate variability, preferably a linear combination of the current pulse frequency P and of the current heart rate variability

Z=A*P+B*HRV
with preferably fixed weighting factors A and B, wherein the heart rate variability is preferably determined by the quotient of the integrals of different frequency ranges obtained from a frequency analysis, preferably a Fourier transform, LFₜₒₜ/HFₜₒₜ,
wherein the status function mentioned above is provided with at least one, preferably an additive correcting value that includes the history of the person at least within the past 0.5 hours, and wherein
the history takes into account a sum P_{Hist} of the measured pulse frequencies P minus a function F₁ at least of the resting heart rate P₀, the sum being weighed with a weighting factor E, with

P_{Hist}=ΣE(t)(P−G*F₁(P₀)).

On the one hand, the features of the invention imply that the values of the current pulse frequency and of the current heart rate variability, each one weighted with respective weighting factors, are added together. This reflects the findings already known from prior art that at a very high pulse frequency the heart rate variability temporarily decreases sharply and thus has a correspondingly reduced significance. On the other hand, in the resting state the pulse frequency has only small significance regarding the stress tolerance, so that then the heart rate variability becomes more important. By providing the above mentioned status function with at least one, preferably additive correcting value that includes the history of the person at least within the past 2 hours, preferably within an interval of about 5 hours to maximally 72 hours, the status value of the stress condition becomes—according to the present invention—considerably more informative.

Thereby, the history is taken into account by means of a sum P_{Hist} of the measured pulse frequencies P minus a function F₁ at least of the resting heart rate P₀, the sum being weighted with a weighting factor E according to

P_{Hist}=E*Σ(P−G*F₁(P₀))

following the method of a moving window (“moving average”). Thereby, E can be a constant, but also a quantity that diminishes linearly with time and which has—assessed from the current point of time—the full value at the beginning of the summation and a negligible value at the end of time. Alternatively, the history can also be implemented by a filter, preferably by a digital lowpass filter that includes the entire history with regard to stress and recovery, which predominantly is a recent history.

In this context, it is reasonable if the function F₁ mentioned above still depends on the age of the person and/or the maximal pulse of the person, preferably as determined by the Conconi test.

According to a second aspect a lowpass filter for the pulse rate history is used instead of the sum function of the pulse history.

According to a third aspect as history a sum of a function F₂ of the ratios LFₜₒₜ/HFₜₒₜ, which sum is weighted with a weighing factor H, according to

HRV_{Hist}=H*Σ(F₂(LFₜₒₜ/HFₜₒₜ))

is taken into account instead of the sum function of the pulse rate history, again according to the method of a moving window (“moving average”).

Thereby, H can again be a constant, but also a quantity that diminishes linearly with time and which has—assessed from the current point of time—the full value at the beginning of the summation and a negligible value at the end of time. Again, the history can alternatively also be implemented by a filter, preferably by a digital lowpass filter that includes the entire history with regard to stress and recovery, but predominantly a recent history.

It is particularly simple and advantageous if the function F₂(LFₜₒₜ/HFₜₒₜ) is a norm function having the values 1 at a current HRV smaller than a first threshold value of a predetermined standardized HRVₙₒᵣₘ, 0 at a current HRV larger than the first threshold value of the predetermined standardized HRVₙₒᵣₘ but smaller than a second threshold value of the predetermined standardized HRVₙₒᵣₘ, and −1 at a current HRV larger than the second threshold value of the predetermined standardized HRVₙₒᵣₘ.

According to a further aspect of the present invention the object is achieved by means of a device for detecting and reporting of a stress condition of a person, the device comprising:
an acquisition device for continuously acquiring the data of at least one body-physiological reference variable, wherein the at least one body-physiological reference variable comprises at least the pulse function of the person,
a processing device for continuously processing the data of the at least one body-physiological reference variable to obtain a current status function of the person,
a comparing device for comparing the acquired current status function of the person with an alert criterion, whereby
the processing device is configured in such a manner that at least a current pulse frequency P and a current heart rate variability HRV are continuously formed from the body-physiological reference variable, whereby
the status function is a function of the current pulse frequency P and of the current heart rate variability, preferably a linear combination of the current pulse frequency P and of the current heart rate variability

Z=A*P+B*HRV
with preferably fixed weighting factors A and B, wherein the heart rate variability is preferably determined by the quotient of the integrals of different frequency ranges obtained from a frequency analysis, preferably a Fourier transform, LFₜₒₜ/HFₜₒₜ,
and the processing device is configured in such manner that the status function mentioned above is provided with at least one, preferably additive correcting quantity that includes the history of the person at least within the past 0.5 hours, and that is configured in such manner that the history includes a sum P_{Hist} of the measured pulse frequencies P minus a function F₁ at least of the resting heart rate P₀, the sum being weighted with a weighting factor E, according to

P_{Hist}=ΣE(t)(P−G*F₁(P₀))
or that the value P_{Hist} is determined by means of a lowpass filter taking into account the previously measured pulse values.

According to a further aspect of the present invention the object is achieved by means of a device for detecting and reporting of a stress condition of a person, the device comprising:
an acquisition device for continuously acquiring the data of at least one body-physiological reference variable, wherein the at least one body-physiological reference variable comprises at least the pulse function of the person,
a processing device for continuously processing the data of the at least one body-physiological reference variable to obtain a current status function of the person,
a comparing device for comparing the thus obtained current status function of the person with an alert criterion, whereby
the processing device is configured in such a manner that at least a current pulse frequency P and a current heart rate variability HRV are continuously formed from the body-physiological reference variable, whereby
the status function is a function of the current pulse frequency P and of the current heart rate variability, preferably a linear combination of the current pulse frequency P and of the current heart rate variability

Z=A*P+B*HRV
with preferably fixed weighting factors A and B, wherein the heart rate variability is preferably determined by the quotient of the integrals of different frequency ranges according from a frequency analysis, preferably a Fourier transform, LFₜₒₜ/HFₜₒₜ,
and the processing device is configured in such manner that the history includes a sum of a function F₂ of the ratios LFₜₒₜ/HFₜₒₜ, which sum is weighted with a weighing factor H, according to

HRV_{Hist}=ΣH(t)F₂(LFₜₒₜ/HFₜₒₜ)
or with a value HRV_{Hist} that is determined by means of a lowpass filter taking into account the previously measured HRV-Values, wherein the function F₂(LFₜₒₜ/HFₜₒₜ) is preferably a norm function having the values 1 at a current HRV smaller than a first threshold value of a predetermined standardized HRVₙₒᵣₘ, 0 at a current HRV larger than the first threshold value of the predetermined standardized HRVₙₒᵣₘ but smaller than a second threshold value of the predetermined standardized HRVₙₒᵣₘ, and −1 at a current HRV larger than the second threshold value of the predetermined standardized HRVₙₒᵣₘ.

The features of the invention according to the two last-mentioned aspects provide a particularly informative device that is relatively simple and reliable as compared to corresponding devices according to the prior art, in particular because it does not determine any superfluous measurement parameters. The device advantageously comprises a display device that is configured in such manner that it can display, preferably graphically, at least the pulse rate, the HRV value during a predetermined or preselected time and also the status function.

According to an aspect of the present invention it is advantageous if the device is configured in such manner that the history includes a sum P_{Hist} of the measured pulse frequencies P minus a function F₁ at least of the resting heart rate P₀, the sum being weighted with a weighting factor E, according to

P_{Hist}=E*Σ(P−G*F₁(P₀))

Thereby, E can be a constant, but also a quantity that diminishes linearly with time and which has—assessed from the current point of time—the full value at the beginning of the summation and a negligible value at the end of time.

Also in this case it is appropriate if the above mentioned function F₁ further depends on the age of the person and/or the maximal pulse of the person, preferably as determined by the Conconi test.

For the function F in the device the following functions are proposed alternatively:

F=(c₁−(Age−20)·c₂)·P₀
with typical values of c₁ [1.01 . . . 6.04], preferably [1.01 . . . 3.00], more preferably 1.2;
or

F=P₀+(P−P₀)·c₃
with typical values of c₃ of [0.10 to 0.50], preferably of [0.15 . . . 0.30], most preferably of 0.20
or

F=Pₘₑₐₙ
wherein Pₘₑₐₙ is a measured, individual average value of the pulse over a representative day.

It is particularly advantageous if the device is configured in such manner that the history also takes into account a sum of a function F₂ of the ratios LFₜₒₜ/HFₜₒₜ, which sum is weighted with a weighting factor H, according to

HRV_{Hist}=H*Σ(F₂(LFₜₒₜ/HFₜₒₜ))

Thereby, H can be a constant, but also a quantity that diminishes linearly with time and which has—assessed from the current point of time—the full value at the beginning of the summation and a negligible value at the end of time.

It is particularly simple and advantageous if the function F₂(LFₜₒₜ/HFₜₒₜ) is a norm function having the values 1 at a current HRV smaller than a first threshold value of a predetermined standardized HRVₙₒᵣₘ, 0 at a current HRV larger than the first threshold value of the predetermined standardized HRVₙₒᵣₘ, but smaller than a second threshold value of the predetermined standardized HRVₙₒᵣₘ, and −1 at a current HRV larger than the second threshold value of the predetermined standardized HRVₙₒᵣₘ. Again, the history can also be implemented alternatively as a filter, preferably a digital lowpass filter that takes into account the entire history regarding stress and recovery, which predominantly is a recent history.

The elements mentioned above and claimed, which shall be used according to the invention, as well as those described in the following exemplary embodiments, are not subject to any particular limitations in terms of their size, shape, use of material and technical design, so that the selection criteria known in the respective application field can be adopted without any restrictions. In particular, the method of the present invention is not intended to assess the health status or the pathological status of the person.

### MODES FOR CARRYING OUT THE INVENTION

The device according to the invention comprises, according to a preferred exemplary embodiment of the invention, a measuring device for detecting the pulse rate and the values that are necessary for calculating heart rate variability. In the present case this is a pulse measuring sensor, but alternatively it can also be an electrical sensor for measuring electric cardiographic measurement values, as well as a display device. Moreover, the device comprises an interface for the input of person-related parameters, which are particularly needed for the detection of the history to be used according to the invention. A key component of the device is a computing device that controls the necessary acquisition of the measurement data, processes the measurement data in their necessary digital form, carries out the data processing and controls the display.

The proposed device is generally used according to the following scheme:

Firstly, a parametrization is performed, which obviously is required only once for every person. The parameterization comprises data on age, sex, and if necessary any other correcting factors, if such shall be used for fine tuning.

Moreover, a step shall be performed that will henceforth be called calibration and that has to be performed periodically, e.g. annually on the occasion of an aptitude test. In this process are determined e.g. the resting heart rate p₀—e.g. in a relaxed state in the morning before breakfast or stress-relieved after 5′ lying down—as well as—if this option is activated—the maximal heart rate pₘₐₓ—e.g. by means of the Conconi test or of a similar stress test. Moreover, a normalized value of the heart rate variability HRVₙₒᵣₘ is determined as base value in a resting state. If moreover e.g. the anaerobic threshold shall also be included in the function, this can also be determined and entered.

It is intended that the device shall be worn in all actions. This results in an operation with a turning on about at least 2 min before the action, although for a complete detection of the history it is obviously better to do the activation several hours before the action. Turning off should also not be done immediately after the effort—if possible—so that relaxation data can be detected (relaxation curve).

The analysis is carried out by the computing device. In the specified exemplary embodiment the display comprises a display of the continuously collected data that depends on the use, e.g. with a temporal display of 10 values per second for the value HRV, but also an averaged data display as well as an analysis of the value HRV by means of a fast Fourier transform FFT with a window of about 2 minutes.

The calculation of a status value (stress value) in the present exemplary embodiment is a function of the pulse rate P and the heart rate variability HRV, and also of the history of the two values (pₕᵢₛₜ, HRVₕᵢₛₜ). Moreover, in the present exemplary embodiment it is contemplated to carry out adaptations to the data of the person, which adaptations are made on the occasion of cyclic calibrations. The history serves the purpose of taking into account previous actions and should include whether a person was already previously exposed to stress (reduction of the performance capability and of the duration of the action, respectively). The status value being used is calculated according to

Stress=A·P+B·Pₕᵢₛₜ+C·HRV+D·HRVₕᵢₛₜ

p: current pulse

HRV: current value of the heart rate variability

Pₕᵢₛₜ: pulse history

HRVₕᵢₛₜ: HRV history

Thereby,

$${P}_{hist} = {\sum }_{- 72 h}^{- 2 min} E ( t ) \cdot ( p - G \cdot F ( {P}_{0} , {P}_{max} , Alter , ... ) )$$

is used with

P₀: resting heart rate, determined e.g. after getting up, before breakfast

Pₘₐₓ: maximal pulse, determined e.g. by the Conconi test

As a simple function F, the device allows selection between different functions, namely

F=(c₁−(Alter−20)·c₂)·P₀
with typical values of c₁ [1.01 . . . 6.04], preferably [1.01 . . . 3.00], most preferably 1.2;
or

F=P₀+(Pₘₐₓ−P₀)·c₃
with typical values of c₃ of [0.10 to 0.50], preferably of [0.15 . . . 0.30], most preferably of 0.20
or

F=^{P}ₘₑₐₙ
wherein Pₘₑₐₙ is a measured, individual average value of the pulse over a representative day.

However, other functions can be selected which include further history- and person-related values, e.g. sex, body size values such as body mass index, etc.

The interval between two heart beats is usually defined as the time between the onsets of two contractions of the cardiac chambers (R wave). Therefore, the distance between two R waves is denoted as RR interval. The real RR intervals vary around the mean heart rate, wherein these variations can even change from beat to beat; this is termed as variability of the cardiac frequency, or heart rate variability (HRV).

There are various characteristic values that are useful for the analysis in the stress sensor, but in the present exemplary embodiment the quotient of the integrals of different frequency ranges obtained from a Fourier transform is used

$$HF ( 0.18 \text{-} 0.40 Hz )$$

$$LF ( 0.04 \text{-} 0.15 Hz )$$

$$HRV = \frac{{LF}_{tot}}{{HF}_{tot}} = \frac{{\int }_{0.04}^{0.15} FFT ( p ) }{{\int }_{0.18}^{0.4} FFT ( p )}$$

Thereby the value HF is mostly attributed to a parasympathetic influence whereas the value LF is attributed to a sympathetic influence.

Alternatively, also a statistical time analysis, namely the variation between the RR intervals (e.g. RMSSD or pNN50) can be used. The device is configured in such manner that the above option can be chosen.

In both cases lower values are worse than higher values because larger variations are indicative of a healthy heart and indicate a normal interplay between the sympathetic and parasympathetic nervous system.

According to the present exemplary embodiment the Fourier transform is carried out in the device by means of the RR data over an interval of about 2 minutes. However, this can be adapted depending on the particular application with the object to obtain a not too large latency but nonetheless sufficient datapoints for a significant value. The calculation of the HRV is carried out according to known algorithms for a fast Fourier transform.

The calculation of the heart rate variability is carried out in the present exemplary embodiment as follows: The norm value for the heart rate variability HRVₙₒᵣₘ for the person is determined on the occasion of the calibration. In general the value lies between 1.5 and 2.0.

$${HRV}_{hist} = H \cdot {\sum }_{- 72 h}^{- 2 min} F ( HRV ) ; HRV = \frac{{LF}_{tot}}{{HF}_{tot}}$$

Example for Function F

1: HRV<d₁·HRVₙₒᵣₘ

F=0: d₁·HRVₙₒᵣₘ<HRV<d₂·HRVₙₒᵣₘ

−1: HRV>d₂·HRVₙₒᵣₘ
wherein d₁<=d₂ and d₁ is in a range of [0.2 . . . 0.6], preferably [0.3 . . . 0.5] and most preferably of 0.4, and d₂ is in the range of [0.5 . . . 0.9], preferably [0.55 . . . 0.75] and most preferably of 0.65.

Examples are shown hereinbelow for subjects

| | Age | p₀ | pₘₐₓ | HRVₙₒᵣₘ |
|---|---|---|---|---|
| A | 20 | 60 | 195 | 2.5 |
| B | 50 | 75 | 170 | 2 |

| | Subject A | | | | Subject B | | | |
|---|---|---|---|---|---|---|---|---|
| | pₕᵢₛₜ | pₕᵢₛₜ | | | pₕᵢₛₜ | pₕᵢₛₜ | | |
| p | V1 | V2 | HRV 1) | HRVₕᵢₛₜ | V1 | V2 | HRV 1) | HRVₕᵢₛₜ |
| 60 | −12.0 | −20.3 | 2.5 | −1 | −7.5 | −29.3 | 2.00 | −1 |
| 105 | 33.0 | 24.8 | 2.0 | −1 | 37.5 | 15.8 | 1.25 | 0 |
| 130 | 58.0 | 49.8 | 1.5 | 0 | 62.5 | 40.8 | 0.50 | 1 |
| 165 | 93.0 | 84.8 | 0.5 | 1 | 97.5 | 75.8 | 0.15 | 1 |

V 1) F = (1.2 − (Age − 20) · 0.01) · P₀
V2 F = P₀ + (Pₘₐₓ − P₀) · 0.15

1. A method for detecting and reporting of a stress condition of a person, wherein the method comprises the following steps:
continuously acquiring the data of at least one body-physiological reference variable, wherein the at least one body-physiological reference variable comprises at least the pulse function of the person,
continuously processing the data of the at least one body-physiological reference variable to obtain a current status function of the person,
comparing the current status function of the person thus obtained with an alert criterion, whereby
at least a current pulse frequency P and a current heart rate variability HRV are continuously obtained from the body-physiological reference variable, whereby
the status function is a function of the current pulse frequency P and of the current heart rate variability, preferably a linear combination of the current pulse frequency P and of the current heart rate variability
Z=A*P+B*HRV
with preferably fixed weighting factors A and B, wherein the heart rate variability is preferably determined by the quotient of the integrals of different frequency ranges obtained from a frequency analysis, preferably a Fourier transform, LFₜₒₜ/HFₜₒₜ,
wherein said status function is provided with at least one, preferably additive, correcting value that includes the history of the person at least within the past 0.5 hour, and wherein
the history takes into account a sum P_{Hist} of the measured pulse frequencies P minus a function F₁ at least of the resting heart rate P₀, the sum being weighed with a weighting factor E, with
P_{Hist}=ΣE(t)(P−G*F₁(P₀))

2. The method according to claim 1, wherein the function F₁ further depends on the age of the person and/or the maximal pulse of the person, preferably as determined by the Conconi test.

3. A method for detecting and reporting of a stress condition of a person, wherein the method comprises the following steps:
continuously acquiring the data of at least one body-physiological reference variable, wherein the at least one body-physiological reference variable comprises at least the pulse function of the person,
continuously processing the data of the at least one body-physiological reference variable to obtain a current status function of the person,
comparing the current status function of the person thus obtained with an alert criterion, whereby
at least a current pulse frequency P and a current heart rate variability HRV are continuously obtained from the body-physiological reference variable, whereby
the status function is a function of the current pulse frequency P and of the current heart rate variability, preferably a linear combination of the current pulse frequency P and of the current heart rate variability
Z=A*P+B*HRV
with preferably fixed weighting factors A and B, wherein the heart rate variability is preferably determined by the quotient of the integrals of different frequency ranges obtained from a frequency analysis, preferably a Fourier transform, LFₜₒₜ/HFₜₒₜ,
wherein said status function is provided with at least one, preferably additive, correcting value that includes the history of the person at least within the past 0.5 hours, and wherein
the value P_{Hist} is determined by means of a lowpass filter taking into account the previously measured pulse values.

4. A method for detecting and reporting of a stress condition of a person, wherein the method comprises the following steps:
continuously acquiring the data of at least one body-physiological reference variable, wherein the at least one body-physiological reference variable comprises at least the pulse function of the person,
continuously processing the data of the at least one body-physiological reference variable to obtain a current status function of the person,
comparing the current status function of the person thus obtained with an alert criterion, whereby
at least a current pulse frequency P and a current heart rate variability HRV are continuously formed from the body-physiological reference variable, whereby
the status function is a function of the current pulse frequency P and of the current heart rate variability, preferably a linear combination of the current pulse frequency P and of the current heart rate variability
Z=A*P+B*HRV
with preferably fixed weighting factors A and B, wherein the heart rate variability is preferably determined by the quotient of the integrals of different frequency ranges obtained from a frequency analysis, preferably a Fourier transform, LFₜₒₜ/HFₜₒₜ,
wherein the status function mentioned above is provided with at least one, preferably additive, correcting value that includes the history of the person at least within the past 0.5 hours, and wherein
the history includes a sum of a function F₂ of the ratios LFₜₒₜ/HFₜₒₜ, which sum is weighted with a weighing factor H according to
HRV_{Hist}=ΣH(t)F₂(LFₜₒₜ/HFₜₒₜ)

5. A method for detecting and reporting of a stress condition of a person, wherein the method comprises the following steps:
continuously acquiring the data of at least one body-physiological reference variable, wherein the at least one body-physiological reference variable comprises at least the pulse function of the person,
continuously processing the data of the at least one body-physiological reference variable to obtain a current status function of the person,
comparing the current status function of the person thus obtained with an alert criterion, whereby
at least a current pulse frequency P and a current heart rate variability HRV are continuously formed from the body-physiological reference variable, whereby
the status function is a function of the current pulse frequency P and of the current heart rate variability, preferably a linear combination of the current pulse frequency P and of the current heart rate variability
Z=A*P+B*HRV
with preferably fixed weighting factors A and B, wherein the heart rate variability is preferably determined by the quotient of the integrals of different frequency ranges obtained from a frequency analysis, preferably a Fourier transform, LFₜₒₜ/HFₜₒₜ,
wherein the status function mentioned above is provided with at least one, preferably additive, correcting value that includes the history of the person at least within the past 0.5 hours, and wherein
the history further includes a value HRV_{Hist} that is weighted with a weighting factor H, which value is determined by means of a low-pass filter taking into account the previously measured HRV-values.

6. The method according to claim 4, wherein the function F₂(LFₜₒₜ/HFₜₒₜ) is a norm function having the values 1 at a current HRV smaller than a first threshold value of a predetermined standardized HRVₙₒᵣₘ, 0 at a current HRV larger than the first threshold value of the predetermined standardized HRVₙₒᵣₘ but smaller than a second threshold value of the predetermined standardized HRVₙₒᵣₘ, and −1 at a current HRV larger than the second threshold value of the predetermined standardized HRVₙₒᵣₘ.

7. A device for detecting and reporting of a stress condition of a person, comprising:
an acquisition device for continuously acquiring the data of at least one body-physiological reference variable, wherein the at least one body-physiological reference variable comprises at least the pulse function of the person,
a processing device for continuously processing the data of the at least one body-physiological reference variable to obtain a current status function of the person,
a comparing device for comparing the acquired current status function of the person with an alert criterion, whereby
the processing device is configured in such a manner that at least a current pulse frequency P and a current heart rate variability HRV are continuously formed from the body-physiological reference variable, whereby
the status function is a function of the current pulse frequency P and of the current heart rate variability, preferably a linear combination of the current pulse frequency P and of the current heart rate variability
Z=A*P+B*HRV
with preferably fixed weighting factors A and B, wherein the heart rate variability is preferably determined by the quotient of the integrals of different frequency ranges obtained from a frequency analysis, preferably a Fourier transform, LFₜₒₜ/HFₜₒₜ,
and the processing device is configured in such manner that said status function is provided with at least one, preferably additive correcting value that includes the history of the person at least within the past 0.5 hours, and that is configured in such manner that the history includes a sum P_{Hist} of the measured pulse frequencies P minus a function F₁ at least of the resting heart rate P₀, the sum being weighted with a weighting factor E, according to
P_{Hist}=ΣE(t)(P−G*F₁(P₀))
or that the value P_{Hist} is determined by means of a lowpass filter taking into account the previously measured pulse values.

8. The device according to claim 7, comprising a display device, wherein the display device is configured in such manner that it can display, preferably graphically, at least the pulse rate, the HRV value during a predetermined or preselected time, and also the status function.

9. The device according to claim 7, wherein said function F₁ further depends on the age of the person and/or the maximal pulse of the person, preferably as determined by the Conconi test.

10. The device according to claim 9, wherein the function
F=(c₁−(Age−20)·c₂)·P₀
with values of c₁ [1.01 . . . 6.04], preferably [1.01 . . . 3.00], most preferably 1.2.

11. The device according to claim 9, wherein the function
F=P₀+(PₘₐₓP₀)·c₃
with values of c₃ of [0.10 to 0.50], preferably of [0.15 . . . 0.30], most preferably 0.20.

12. The device according to claim 9, wherein
F=, wherein Pₘₑₐₙ is a measured, individual average value of the pulse over a representative day.

13. A device for detecting and reporting of a stress condition of a person comprising:
an acquisition device for continuously acquiring the data of at least one body-physiological reference variable, wherein the at least one body-physiological reference variable comprises at least the pulse function of the person,
a processing device for continuously processing the data of the at least one body-physiological reference variable to obtain a current status function of the person,
a comparing device for comparing the acquired current status function of the person with an alert criterion, whereby
the processing device is configured in such manner that at least a current pulse frequency P and a current heart rate variability HRV are continuously formed from the body-physiological reference variable, whereby
the status function is a function of the current pulse frequency P and of the current heart rate variability, preferably a linear combination of the current pulse frequency P and of the current heart rate variability
Z=A*P+B*HRV
with preferably fixed weighting factors A and B, wherein the heart rate variability is preferably determined by the quotient of the integrals of different frequency ranges obtained from a frequency analysis, preferably a Fourier transform, LFₜₒₜ/HFₜₒₜ,
and the processing device is configured in such manner that the history includes a sum of a function F₂ of the ratios LFₜₒₜ/HFₜₒₜ, which sum is weighted with a weighing factor H, according to
HRV_{Hist}=ΣH(t)F₂(LFₜₒₜ/HFₜₒₜ)
or with a value HRV_{Hist} that is determined by means of a lowpass filter taking into account the previously measured HRV-Values, wherein the function F₂(LFₜₒₜ/HFₜₒₜ) is preferably a norm function having the values 1 at a current HRV smaller than a first threshold value of a predetermined standardized HRVₙₒᵣₘ, 0 at a current HRV larger than the first threshold value of the predetermined standardized HRVₙₒᵣₘ but smaller than a second threshold value of the predetermined standardized HRVₙₒᵣₘ, and −1 at a current HRV larger than the second threshold value of the predetermined standardized HRVₙₒᵣₘ.

14. The method according to claim 5, wherein the function F₂(LFₜₒₜ/HFₜₒₜ) is a norm function having the values 1 at a current HRV smaller than a first threshold value of a predetermined standardized HRVₙₒᵣₘ, 0 at a current HRV larger than the first threshold value of the predetermined standardized HRVₙₒᵣₘ but smaller than a second threshold value of the predetermined standardized HRVₙₒᵣₘ, and −1 at a current HRV larger than the second threshold value of the predetermined standardized HRVₙₒᵣₘ.
